(19) United States
(12) Patent Application Publication
(10) Pub. No.: US 2010/0228172 A1
(43) Pub. Date: Sep. 9, 2010

(54) Toe protectors, shrouds, and protective covers for shrouds

(76) Inventors: Kent Biddinger, Midland, MI (US); Chanseol Chung, Cuyahoga, OH (US); Jeanette V. D'Agostino, Livingston, MI (US); Matthew Hanson, Cuyahoga, OH (US); Linda M. Horbal, Midland, MI (US)

Correspondence Address: MCKELLAR IP LAW, PLLC, 784 SOUTH POSEYVILLE ROAD, MIDLAND, MI, 48640, US

(21) Appl. No.: 12/660,650

(22) Filed: Mar. 2, 2010

Related U.S. Application Data
(60) Provisional application No. 61/209,192, filed on Mar. 4, 2009.

(51) Int. Cl. A61F 5/00

(52) U.S. Cl. 602/30

(57) ABSTRACT

Toe protectors for patients that have had toe surgery or otherwise have a need to protect the toes. The toe protectors are easy to ship or transport, are easy to assemble and apply to the foot, are easy to remove for ascertaining the progress of the surgery or injury, are economical to manufacture, and are sturdy and easy to clean.

This application is a utility application claiming priority from U.S. Provisional application Ser. No. 61/209,192, filed on Mar. 4, 2009.

The inventions described herein deal with toe protectors, shrouds, and protective covers for shrouds, for patients that have had toe surgery or otherwise have a need to protect the toes. The toe protectors of this invention are easy to assemble and apply to the foot, are easy to remove for ascertaining the progress of the surgery or injury, are economical to manufacture, and are sturdy and easy to clean.

### BACKGROUND OF THE INVENTION

Toe protectors are used to protect the toes of the human foot, mainly after surgery and are also used for protecting broken appendages.

The protectors consist mainly of a front protector that is a solid piece of material with a provisional means to attach them to the foot. Certain of the toe protectors also have a sole or plate on which the protector rests so that the foot does not reach the ground when the patient is walking and these are typically in the form of shoes or slippers.

There is a fracture splint covering disclosed in U.S. Pat. No. 2,949,912 that issue on Aug. 23, 1960 to Shapiro in which there is shown a cup that goes over the toes which is held in place by a strap that reaches around the back of the ankle. This covering has a pedestal to rest the device on.

A similar device can be found in U.S. Pat. No. 3,487,830 that issued to Pruett on Jan. 6, 1970 in which there is shown a cup with a back strap and which also discloses a pedestal that fits under the heel of the foot.

In U.S. Pat. No. 3,643,659 that issued Feb. 22, 1972 to Storer, there is disclosed a solid toe protector that is worn to support bedding or other soft covering. This device also has a heel cup.

Cappelletti, in U.S. Pat. No. 3,832,997 that issued on Sep. 3, 1974 disclosed a device that is intended for combination with a plaster cast of the type which is applied to the foot of a fracture patient and which leaves the toes uncovered.

U.S. Pat. No. 4061,138 that issued on Dec. 6, 1977 to Bernstein discloses a toe protector and foot support for an orthopedic cast that consists of a long flat blade surmounted by a cup.

Chapman, in U.S. Pat. No. 4,177,583 discloses an orthopedic shoe with a forefoot protective guard in the form of a cup.

Brouhard in U.S. Pat. No. 4,454,872 that issued on Jun. 19, 1984 discloses a wrap-around protective device for human toes when the foot is encased in an orthopedic cast. The protector is held in place by shoe laces strapped to the device.

There is disclosed in U.S. Pat. No. 4,566,208 that issued on Jan. 28, 1986 to Shaffner a combination of side and front rails to protect the toes.

Nielsen, et al, in U.S. Pat. No. 5,185,945 that issued on Feb. 16, 1993 shows a universal protective shield for the foot which can be drawn together to form a protective cover. It is noted in the patent that the device may be adapted from a flat template or reformed to fit different shaped foot coverings.

Design patent 391,744 that issued to McMaster on Mar. 10, 1998 discloses a toe protector which is a cup that has a strap that closes around the back of the heel.

Holt et al in U.S. Pat. No. 5,778,565 that issued on Jul. 14, 1998 discloses a protector that has a removable toe piece and Rodi, in U.S. Pat. No. 6,272,771 that issued on Aug. 14, 2001 discloses a semi-rigid arcuate enclosure that is in the form of a shoe and overlays and extends beyond the end of the toes.

Cook, in U.S. Pat. No. 6,514,222 that issued Feb. 4, 2003 discloses a simple open hook shaped protector that is held in place on the foot using a strap over the top of the foot and U.S. Pat. No. 6,802,318 that issued Oct. 12, 2004 to Parker discloses a flexible sheet of plastic that is formed into a U-shape that is held in place on the foot by a strap over the top of the foot.

Woods in U.S. Pat. No. 6,836,980 that issued on Jan. 4, 2006 deals with a toe guard assembly that uses adhesive material to stick tabs, containing a forward cup, to the sides of a shoe apparatus.

None of the prior art devices disclosed Supra are formed from a flat sheet of plastic that is folded origami style to provide a solid, sturdy, toe protector that can be uncoupled to examine the progress of the healing of the toes with the capability of reattaching the toe protector to the foot.

### THE INVENTION

Thus, this invention provides a toe protector, wherein the toe protector comprises a foldable toe cover, the foldable toe cover comprising an integral flat, pre-designed, pre-scored, plastic sheet having a front segment comprised of a flat middle portion having a distal end, there being two lateral side tabs at the distal end.

There is a flat middle segment integrally attached to the flat front segment on the same plane, the middle segment being comprised of a middle portion having lateral side components, wherein each side component is in the form of a tab, the tab comprising a near portion, a middle portion and a distal portion.

There is a flat back segment integrally attached to the flat middle segment on the same plane and flat wedge-shaped connectors integrally attached in the same plane, between each interstices formed by each flat front segment and each flat middle segment and between each flat middle segment and the flat back segment.

In yet another embodiment of this invention there is a cover for protecting a toe portion of a human body wherein the toe portion has in place a toe protector as disclosed Supra, to fit and protect the toe portion and toe protector from liquids and dirt.

The cover comprises a boot-like shroud having a unitary structure, wherein the shroud has a front, two oppositely opposed side walls, a top, a back foldable portion and a bottom having a top and a bottom surface.

The bottom has a reinforced lining surmounting the top surface and the foldable back portion has a terminal end, the terminal end having an opening in it.

There are two strap segments, each fixed to the bottom surface of the bottom, near the back of the shroud such that the two strap segments will fasten to each other over the top of the shroud.

It is also contemplated within the scope of this invention to provide a bottom sole for the cover wherein the sole is manufactured from such materials as semi-rigid foam, rubber, and soft plastics.

Further contemplated within the scope of this invention is a cover in which the straps have a fastener that can be, for example, hook and loop, buttons, snaps, and Laces.

Finally there is another embodiment that is a protective cover for a shroud and toe protector as described Supra, said protective cover having an oversize flexible boot-like configuration that is an integral unit, the cover being used principally to protect bed linens and furniture from dirt and the like that accumulates on the toe protector and boots or casts.

The boot-like cover has a foot portion and a leg portion, wherein the leg portion has a top edge and a fastening means near the top edge of the leg portion.

### BRIEF DESCRIPTION OF THE DRAWINGS

FIG. 1A is full top view of a flat sheet plastic material showing the configuration of the toe protector before folding.

FIG. 1B is a full top view of a flat sheet plastic material showing the configuration of the toe protector before folding, wherein there is shown air holes in the top segment.

FIG. 1C is a full top view of a flat sheet plastic material showing the configuration of the toe protector before folding, wherein there is shown the air holes as decorative flowers.

FIG. 2 is a full side view of the toe protector that has been folded from the flat plastic sheet in an origami style to partially complete the toe protector.

FIG. 3 is a full side view of a fully assembled toe protector.

FIG. 4 is a full top view of the toe protector

FIG. 5 is a fully assembled shroud for the toe protector.

FIG. 6 is a full side view of the boot used to cover and protect the shroud and the protective toe cover of this invention.

### DETAILED DESCRIPTION OF THE INVENTION

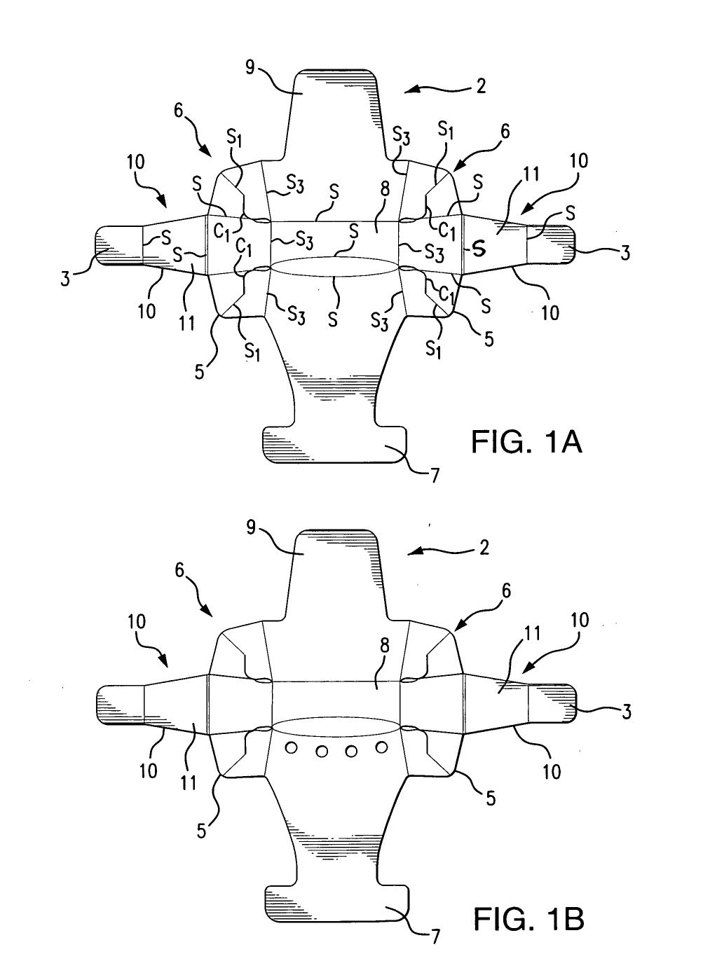

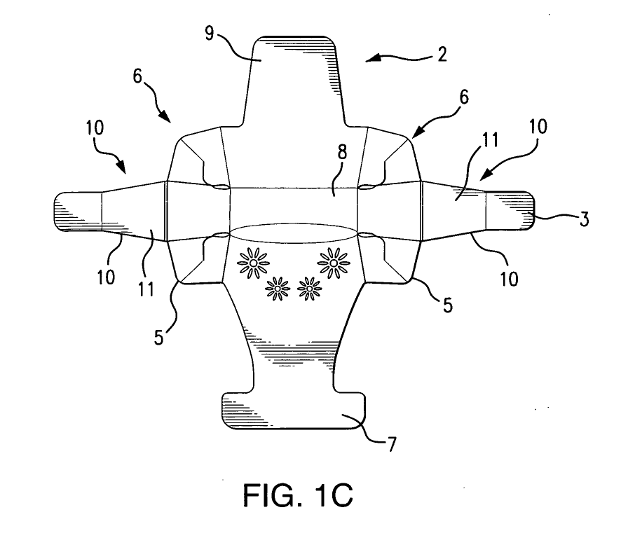

Shown in FIG. 1 is a full top view (inside view) of the flat plastic sheet 1 having the toe protector configuration before folding, from which the toe protector 2 of this invention is fabricated.

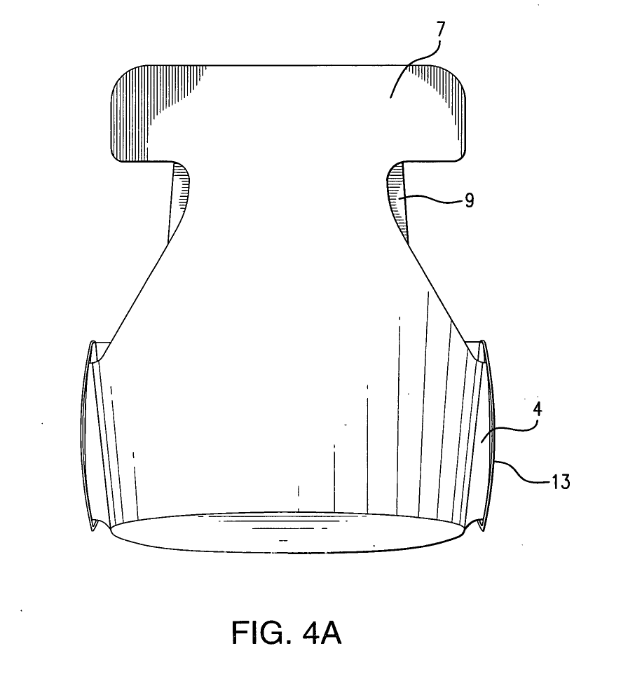

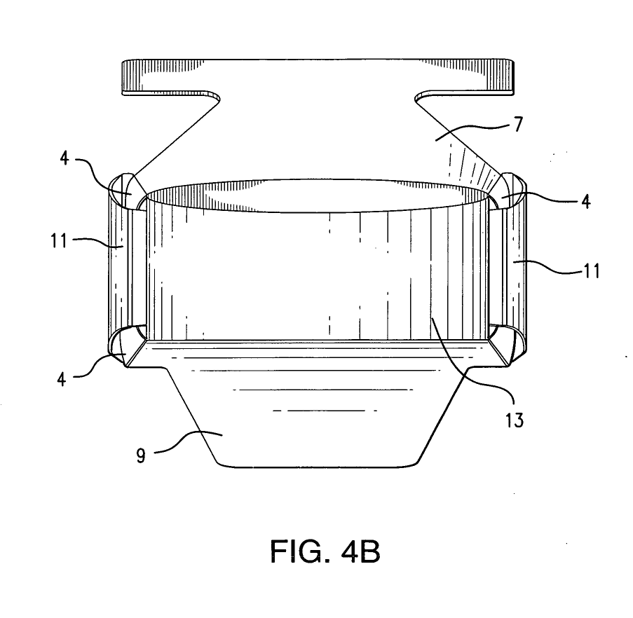

The flat plastic sheet is pre-designed and pre-scored to allow for the foldability of the flat sheet into the toe protector 2. The toe protector 2 is folded origami style and when all of the components are folded into place, the tabs 3 (FIG. 1A) are folded into pockets 4 (FIG. 4B) to hold the toe protector 2 together and in the desired configuration.

Turning now to the flat sheet in FIG. 1A, there is shown by the designation S all of the score marks that are required to allow the foldability of the sheet into the toe protector 2. According to this invention, the flat sheet is folded in the following general manner, it being understood that the order of folding on the pre-scored lines is not significant.

One begins by folding, for example, lines S₁ which are in the wedge segments 5 of the configuration as shown in FIG. 1A. This action forces cut lines C₁ to open up and forces the wedge segments 5 and 6 to fold outwardly along line S₁.

This action gives a partially folded wedge segment 5 between the interstitial space formed by the front segment 7 and the flat middle segment 8 and the wedge segment 6 between the interstitial space formed by the flat middle segment 8 and the flat back segment 9.

Next, for example, the pre-scored lines S₃ are folded such that each flat middle segment 8, along with the wedges 5 and 6, are folded inwardly.

Thereafter, the lateral side components 10 of the flat middle segment 8 are drawn inwardly while the middle portion 11 of each lateral side component 10 is folded outwardly. Further, the distal portion 3 is also folded outwardly.

When the wedge segments 5 and 6 are entirely folded according to that set forth Supra, the flat middle segment 8 and the flat front segment 7, along with the flat back segment 9 are all moved inwardly toward each other, the wedges 5 and 6 can be folded towards each other and essentially laid flat against the outside wall 13 (FIG. 4B) of the middle portion 11 of the middle segment 8.

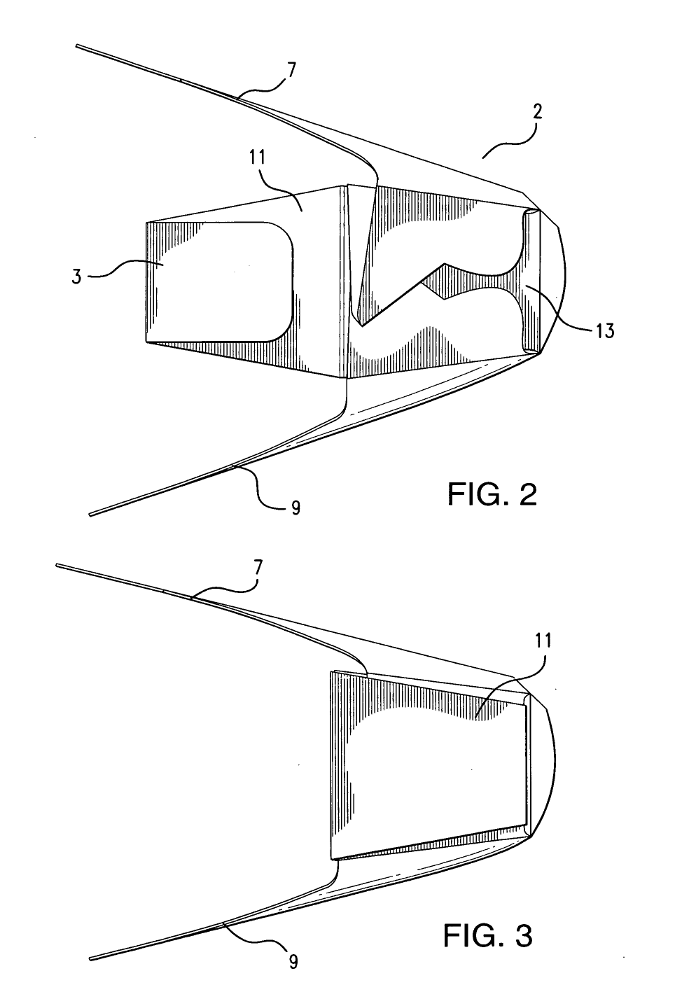

FIG. 2 illustrates the partial folding of the toe protector 2 wherein there is shown distal end 3, the outside wall 13 of the middle portion 11 of the middle segment 8.

While held in that position, the middle portion 11 of the middle segment 8 is folded down over the flattened wedges 5 and 6 as they lay against the outside wall 13 of the middle portion 11 of the middle segment 8.

Thereafter, the distal portion 3 of the middle segment 8 is inserted in the pocket 4 formed thereby. By this means, the toe protector is held in the desired folded position. The fully folded toe protector 2 is shown in FIG. 3 which is a full side view of the device while FIG. 4A is a full top view.

By reversing this procedure, that is, by removing the distal end 3 from the pocket 4, the toe protector 2 can be opened and removed from the foot of the patient. This means, the toe protector 2 can be opened for inspection and then closed, all with ease.

FIG. 4B is a full front view of the toe protector 2 after being fully folded, showing especially the pockets 4 on each side of the device.

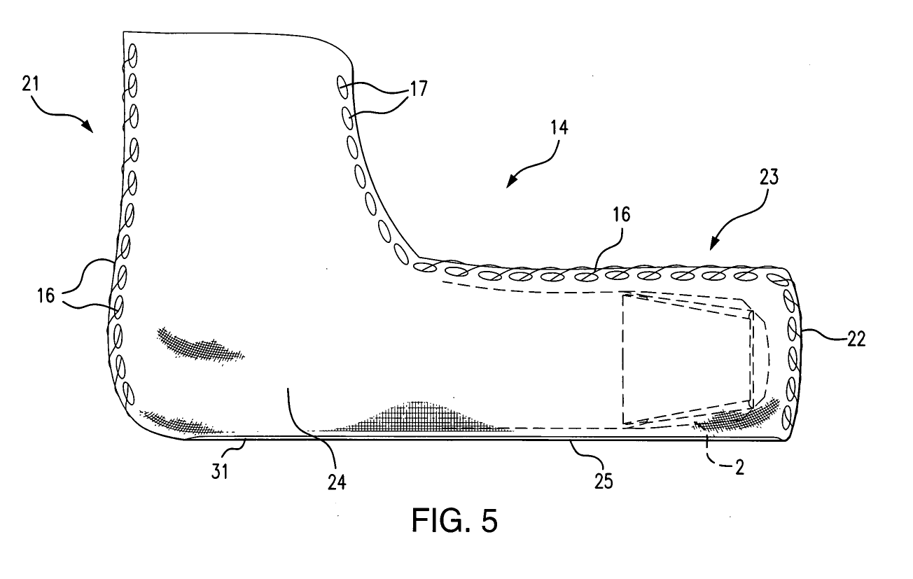

Turning now to FIG. 5, there is shown therein a shroud 14 in combination with a toe protector 2 of this invention showing the toe protector 2 in phantom wherein there is shown stitching 16 for the construction of the shroud 14 and in this Figure, eye holes 17 for a lace to fasten the shroud 14 over the toe protector 2. Also shown are the back 21, the front 22, the top 23, and one side 24, which is identical to the opposite side. There is also a bottom 25.

The shroud 14 is typically manufactured from a soft cloth or fabric such that it can be removed occasionally and washed

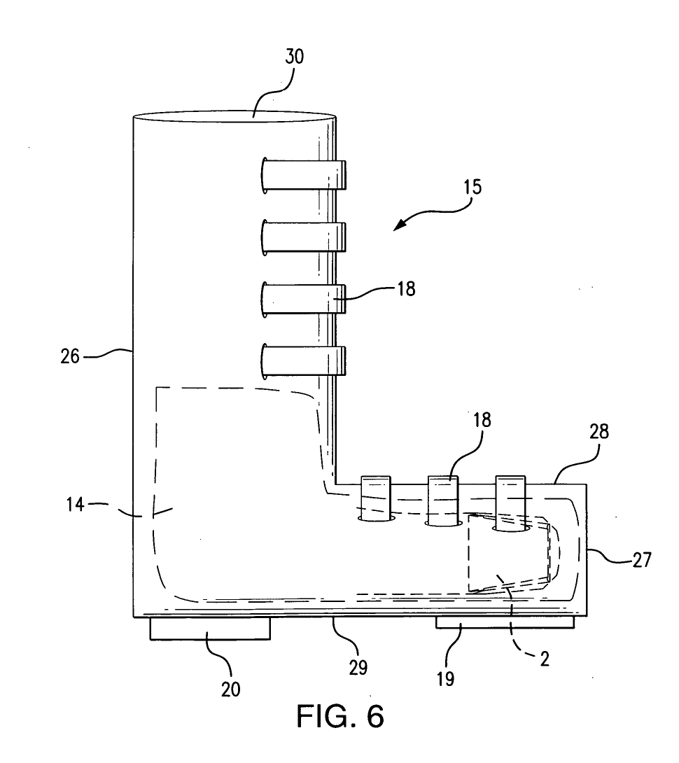

With regard to FIG. 6, there is shown a full walking boot 15 in combination with the shroud 14 and the toe protector 2 wherein the shroud 14 is shown in long dotted lines and the toe protector 2 is shown in short dotted lines, in phantom, wherein there is additionally shown straps 18 for strapping the boot 15 to the leg and foot of the patient, the back 26, the front 27, the top 28, the bottom 29, and the top opening 30. Typically, the boot has a water proof and hard sole 19 and a waterproof and hard heel 20.

What is claimed is:

1. A toe protector, wherein the toe protector comprises a foldable toe cover, the foldable toe cover comprising an integral flat, pre-designed, pre-scored, plastic sheet having:
a. front segment comprised of a flat middle portion having a distal end, there being two lateral side tabs at the distal end thereof;
b. a flat middle segment integrally attached to the flat front segment on the same plane, said middle segment comprised of a middle portion having lateral side components, each side component being in the form of a tab, said tab comprising a near portion, a middle portion and a distal portion;
c. a flat back segment integrally attached to the flat middle segment on the same plane;
d. flat wedge-shaped connectors integrally attached in the same plane, in each interstitial opening formed by the front segment and the flat middle segment and each interstitial opening between the flat middle segment and the flat back segment.
